(19) United States
(12) Patent Application Publication
(10) Pub. No.: US 2022/0079800 A1
(43) Pub. Date: Mar. 17, 2022

(54) ARM SLING APPARATUS

(71) Applicant: Keith M. Baumgarten, Sioux Falls, SD (US)

(72) Inventor: Keith M. Baumgarten, Sioux Falls, SD (US)

(21) Appl. No.: 17/023,841

(22) Filed: Sep. 17, 2020

(51) Int. Cl. A61F 5/37

(52) U.S. Cl. CPC A61F 5/3746

(57) ABSTRACT

A sling apparatus for supporting an arm of a person may include a garment to be worn on the torso, and arm support elements mounted on the garment that are configured to support the arm of the person wearing the garment in at least two positions with respect to the torso. The two positions may include a first position of the arm having the elbow extended with a shoulder associated with the arm of the person being in a neutral rotation orientation, and a second position of the arm having the shoulder associated with the arm being in an adducted, internal rotation position with the elbow flexed.

### BACKGROUND

### Field

The present disclosure relates to rehabilitation body supports and more particularly pertains to a new arm sling apparatus for providing versatility in the positions in which the arm can be supported by the apparatus.

### SUMMARY

In one aspect, the present disclosure relates to a sling apparatus for supporting an arm of a person with respect to a torso of the person, with the arm having an upper arm portion and a lower arm portion. The sling apparatus may include a garment configured to be worn on the torso of the person, with the garment having a front for positioning adjacent to a front of the torso, a back for positioning adjacent to a back of the torso, and at least one lateral side extending between the front and back for positioning adjacent to a side of the torso. The garment may have at least one arm opening in the at least one lateral side for receiving the arm of the person. The sling apparatus may also include arm support elements mounted on the garment and configured to support the arm of the person wearing the garment in at least two positions with respect to the torso. The at least two positions may include a first position of the arm having the elbow extended with a shoulder associated with the arm of the person being in a neutral rotation orientation and a second position of the arm with the elbow flexed and the shoulder associated with the arm being in an adducted, internal rotation position.

There has thus been outlined, rather broadly, some of the more important elements of the disclosure in order that the detailed description thereof that follows may be better understood, and in order that the present contribution to the art may be better appreciated. There are additional elements of the disclosure that will be described hereinafter and which will form the subject matter of the claims appended hereto.

In this respect, before explaining at least one embodiment or implementation in greater detail, it is to be understood that the scope of the disclosure is not limited in its application to the details of construction and to the arrangements of the components set forth in the following description or illustrated in the drawings. The disclosure is capable of other embodiments and implementations and is thus capable of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present disclosure. It is important, therefore, that the claims be regarded as including such equivalent constructions insofar as they do not depart from the spirit and scope of the present disclosure.

The advantages of the various embodiments of the present disclosure, along with the various features of novelty that characterize the disclosure, are disclosed in the following descriptive matter and accompanying drawings.

### BRIEF DESCRIPTION OF THE DRAWINGS

The disclosure will be better understood and when consideration is given to the drawings and the detailed description which follows. Such description makes reference to the annexed drawings wherein:

FIG. 1 is a schematic front view of a new sling apparatus according to the present disclosure.

FIG. 2 is a schematic side view of the sling apparatus, according to an illustrative embodiment.

FIG. 3A is a schematic front view of the sling apparatus worn by a person and supporting an arm of the person in a first position.

FIG. 3B is a schematic front view of the sling apparatus worn by a person and supporting the arm of the person in a second position.

FIG. 3C is a schematic front view of the sling apparatus worn by a person and supporting the arm of the person in a third position.

### DETAILED DESCRIPTION

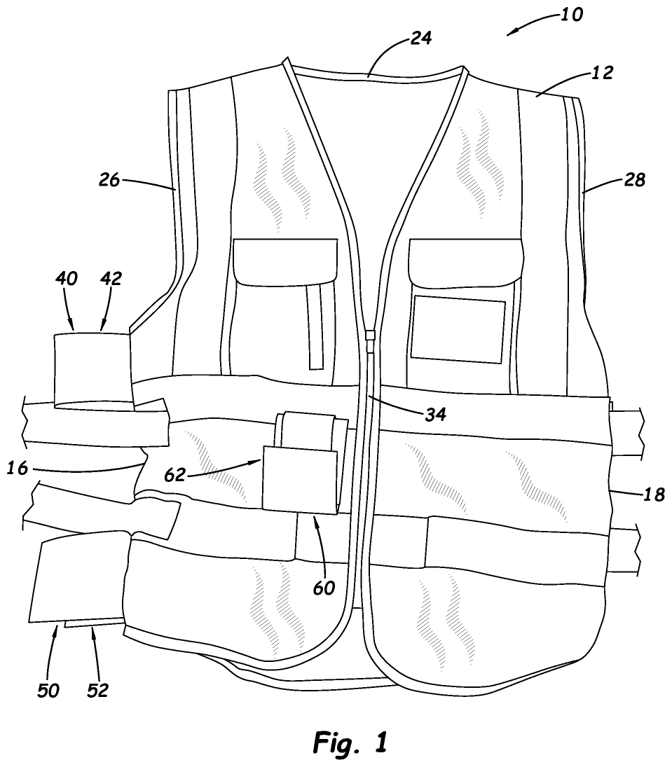

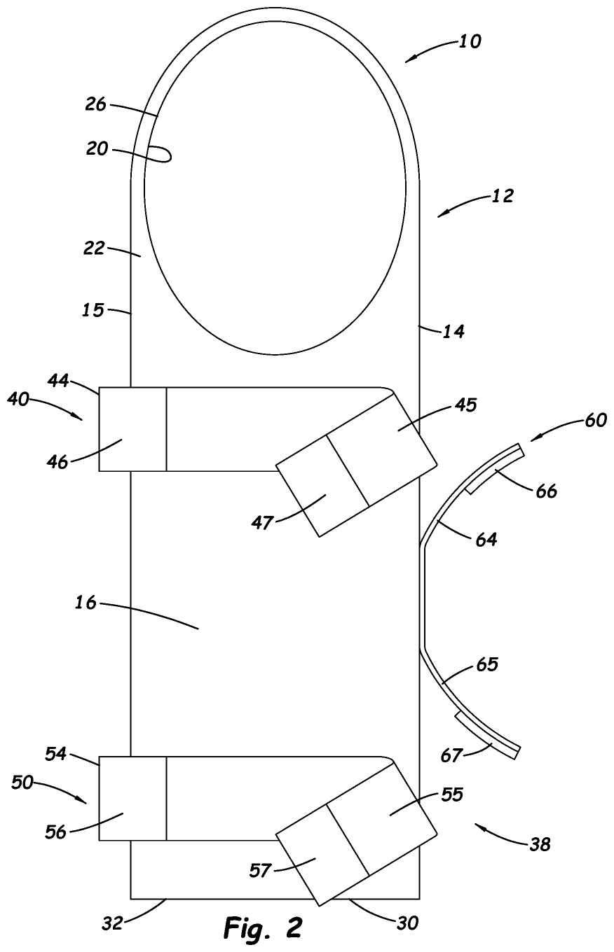

With reference now to the drawings, and in particular to FIGS. 1 through 3 thereof, a new arm sling apparatus embodying the principles and concepts of the disclosed subject matter will be described.

Traditional rehabilitative arm supports, or arm slings, immobilize the elbow of the user of the sling in a flexed position, but the applicant has recognized that the usage of traditional arm slings can be problematic. For example, traditional arm slings promote prolonged elbow flexion which has been shown to increase the pressure within the cubital tunnel through which the ulnar nerve courses, and as a result putting the person at risk for developing symptoms of ulnar neuritis. Moreover, when used inappropriately, traditional arm slings may not support the wrist adequately, causing the sling user to maintain a hyper flexed position of the wrist that puts the patient at risk for developing symptoms of carpal tunnel syndrome.

The applicant has further recognized that distal peripheral neuropathy, which includes cubital tunnel syndrome and carpal tunnel syndrome, is a common complication after shoulder surgery and is likely due to contemporary methods of postoperative shoulder immobilization.

The applicant has developed a novel sling apparatus which maintains the user's arm in an elbow extended position that can be used to minimize the opportunity for the onset of symptoms of ulnar neuritis after shoulder surgery and can be used to treat symptoms of ulnar neuritis that arise de novo. The elbow extended position provided by the sling apparatus of the disclosure may be considered an optimal choice for use during sleep once the patient is comfortable sleeping in the supine position. In addition, when the sling is used by the patient in the supine position, the patient's wrist is immobilized against the torso and functionally maintains the wrist in the neutral position. As a result, the sling apparatus of the disclosure may also prevent the onset of carpal tunnel symptoms after shoulder surgery.

Further, the applicant has recognized that the majority of arm sling designs currently utilized will maintain the user's arm in complete internal rotation (for anti-rotation and internal rotation sling designs) to partial internal rotation (for abduction sling designs), and that very few of the currently utilized sling designs precisely maintain the arm in a neutral position. Although neutral rotation arm slings are designed to maintain the arm in neutral rotation, these designs often allow the immobilized arm to rotate into an internal rotated position. Moreover, there does not appear to be a consensus as to which type of sling (e.g., abduction sling vs. an internal rotation sling) is optimal for a patient after shoulder surgery, although some advantage has been suggested in using a neutral rotation sling over an internal rotation sling to provide improvements in range of motion and decreased night pain after anatomic total shoulder arthroplasty. The applicant has recognized that while further studies need to be performed regarding the optimal positioning after shoulder surgery, there appear to be benefits to immobilizing the arm in either a neutral rotation position or a position with mild external rotation.

The sling apparatus of the present disclosure provides the ability to immobilize the user's arm in the extended position with the shoulder being maintained in a neutral rotation position which has been shown to be beneficial in patients undergoing anatomic total shoulder arthroplasty and may have benefits after other shoulder surgeries such as arthroscopic rotator cuff repair.

Advantageously, the sling apparatus of the present disclosure is a dual positioning sling which provides the patient and the physician with a choice between more than one position to support the arm. As a result, the supportive position may be changed for different conditions or situations. For example, if the shoulder girdle of the user fatigues when the arm has been maintained in the elbow extension/shoulder neutral rotation position (e.g. due to the weight of the arm not being supported by the sling apparatus), the user can transition his or her arm to the internal rotation position which supports the weight of the arm more substantially than the elbow extension/shoulder neutral rotation position.

As a further example, immediately after surgery, when the patient has little motor control of his or her arm secondary to a peripheral nerve block, or when the patient is in a seated position, the internal rotated position may be more beneficial. Moreover, when the patient is seated, it may be very uncomfortable to have the elbow extended with the arm immobilized at the side.

The applicant has also recognized that immobilization of the arm after surgery using traditional arm slings can make it difficult to perform activities of daily living and light work activities (like computer work). However, arm slings able to provide support in a neutral rotation can permit better work performance and patient satisfaction as compared to slings which only provide support in an internal rotation position.

Advantageously, the sling apparatus of the present disclosure may be used as a functional sling when complete arm immobilization (e.g., in an extension position or in an internal rotation position) may not be desired. The sling apparatus permits the upper arm above the elbow to be immobilized in the adducted position and held against the torso, thereby protecting the shoulder, while allowing free motion at the elbow, wrist and hand of the arm and further allowing self-limited active internal and external rotation of the shoulder. In more protected and safe environments, the sling apparatus of the present disclosure permits patients to handle light objects, perform computer and sedentary work, feed themselves, and perform hygiene tasks, which are functions that are difficult to perform utilizing contemporary slings.

Further, the applicant recognizes that the application of low levels of controlled force may be beneficial to the healing of the rotator cuff, and that complete removal of the load from the rotator cuff may be detrimental to rotator cuff healing, possibly suggesting that it may be beneficial to not use any sling immobilization or to allow early active range of motion after rotator cuff repair surgery. The sling apparatus of the present disclosure may provide patients with more functional use of their postoperative extremity compared to traditional arm slings while still providing protection to the operative arm compared to not utilizing a sling at all.

Still further, the applicant has recognized that traditional arm slings often utilize a strap that traverses the neck and trapezius area of the user, which may contribute to neck and trapezial pain, and may even cause carotid hypersensitivity syndrome that can result in dizziness and syncope and spinal accessory nerve palsy. The sling apparatus of the present disclosure utilizes a garment which may engage a relatively large area of the user's torso and thus does not concentrate pressure on the user's neck, and consequently is less likely to cause neck pain or the rare complications that have been associated with a sling that utilizes a strap that courses around the neck. Moreover, while some patients have difficulty learning how to correctly don and maintain the appropriate arm position in traditional arm slings, as the wearing of a sling is not familiar or intuitive, the garment of the sling apparatus of the present disclosure may be similar to a vest which is familiar to the patient and is difficult to wear incorrectly.

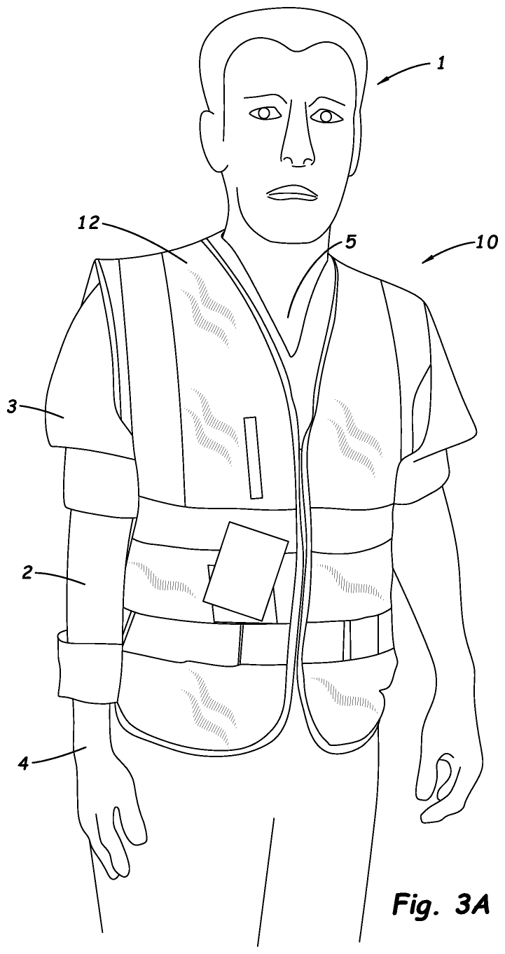

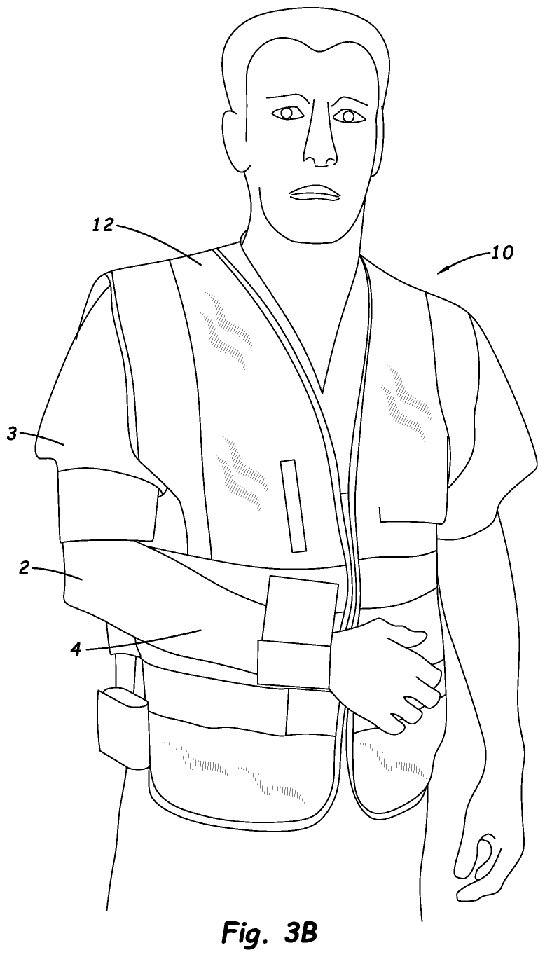

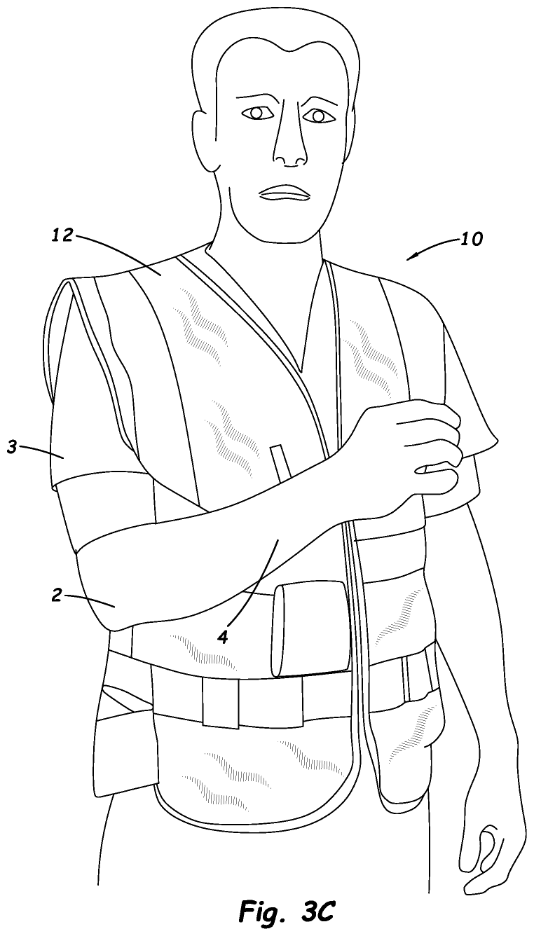

In one aspect, the present disclosure relates to a sling apparatus 10 for supporting an arm 2 of a person 1, which may include an upper arm portion 3 and a lower arm portion 4 with an elbow in between the portions 3, 4. The sling apparatus 10 may support the arm in at least two positions of the arm with respect to the torso 5 of the person. In some embodiments, a first position of the arm 2 of the person may have the elbow of the arm extended with the shoulder of the person in a neutral rotation orientation (see FIG. 3A), which may have the lower arm portion of the person secured to the lateral aspect of the torso of the person, and a second position may have the arm of the person in an adducted, internal rotation position of the arm (see FIG. 3B), which may have the elbow of the person flexed with the lower arm portion secured to the ventral or anterior aspect of the torso of the person. In some implementations, a third position of the arm 2 has the upper arm portion 3 constrained in a position adjacent to the torso 5, but the lower arm portion 4 is unconstrained and generally free to move relative to the torso (see FIG. 3B).

The sling apparatus 10 may include a garment 12 which is configured to be worn by the person 1 using the apparatus 10 who may be a patient under medical supervision. The garment 12 may be configured to be worn on the torso 5 of the person such that the garment 12 covers, or substantially covers, the torso from the shoulders down to a point proximate to the waist of the person. In greater detail, the garment 12 includes a front 14 for positioning adjacent to the front of the person's torso, and a back 15 for positioning adjacent to the back of the person's torso. The lateral sides 16, 17 of the garment 12 may extend between the front 14 and back 15 for positioning adjacent to sides of the person's torso. The garment 12 has an inner extent or surface 20 for positioning against (or in close proximity to) the person's torso, and an outer surface 22 may be located opposite of the inner surface 24 on the garment for positioning away from the person's torso.

In further detail, the garment 12 may have a neck opening 24 which extends through the garment generally between the front 14 and back 15 for receiving the neck of the person wearing the garment duck opening 24. A pair of arm openings 26, 28 may extend through the garment 12 at the lateral sides 16, 17 between the front and back of the garment for receiving the respective arms of the person. A torso opening 30 may be located on the garment 12 opposite of the neck opening 24 to permit a portion of the person's torso 3 to extend out of the garment 12. Illustratively, the garment 12 may comprise a vest garment which generally lacks sleeves for receiving the arms of the person wearing the garment. A slit 34 may be formed on the garment 12 to facilitate donning and doffing the garment on the torso of the person. The slit 34 may be positioned on, and extend across, the front 14 of the garment, and may extend from the neck opening 24 to the torso opening 30 to permit the garment to effectively be opened up to simplify placing the garment on the torso and removing the garment from the torso. Suitable closure means, such as a zipper closure, may be mounted along the edges of the material forming the slit to selectively join the edges forming the slit. Optionally, the slit 34 may be omitted in some embodiments of the garment 12. Further, belts or straps may be utilized on the garment to further secure the garment in place on the torso of the user.

The sling apparatus 10 may also include arm support elements 38 configured to support the arm 2 of the person 1 wearing the garment 12, and the support elements 38 may be configured to support the arm in at least two positions of the arm, such as a neutral rotation orientation position and an adducted, internal rotation position. In some embodiments, the arm support elements 38 may include a plurality of loops for removably receiving portions of the arm 2 that are desired to be supported by the apparatus 10. In addition to supporting portions of the arm, the plurality of loops may also constrain portions of the arm in each of the at least two positions.

The plurality of loops of the arm support elements 38 may include a first loop 40 which is configured for removably receiving the upper arm portion 3 of the arm of the person. The first loop 40 may be mounted on the outer surface 22 of the garment 12 and secured to the garment in any suitable manner, such as, for example, by sewing or otherwise bonding a portion of the loop 40 to the material of the garment. The first loop 40 may be positioned below a first one of the arm openings 26 of the garment, and may be positioned between the first arm opening and the lower edge 32 of the garment forming the torso opening 30. The first loop 40 may be positioned adjacent to the first arm opening 26 in a location suitable to receive the upper arm portion 3 of the person when the garment is worn by the person.

The first loop 40 may define a first passage 42 through which the upper arm portion 3 is insertable, and the first passage may extend along a substantially vertically-oriented axis when the garment 12 is worn by a standing person such that the upper arm portion 3 is held in a substantially vertical orientation in association with the torso 5.

In some embodiments, the first loop 40 may be configured to open to receive the upper arm portion 3 into, and release the upper arm portion from, the loop 40. The first loop 40 may also be configured to close to encircle and embrace the upper arm portion 3 to secure the arm portion 3 against movement out of the first loop. Illustratively, the first loop 40 may include a pair of first flaps 44, 45 which are joinable together to close the loop 40, and which are releasable from each other to open the loop 40. The first flaps 44, 45 may have connecting elements 46, 47 thereon to permit releasable connection of the first flaps together, as well as to permit release of the first flaps from each other. Optionally, the first connecting elements 46, 47 may comprise a hook and loop fasteners, such as fasteners sold under the VELCRO trademark.

The plurality of loops of the arm support elements 38 may further include a second loop 50 which is configured for removably receiving the lower arm portion 4 of the arm of the person. The second loop 50 may be mounted on the outer surface 22 of the garment 12 by securing the loop 50 to the garment using any suitable means. The second loop 50 may be positioned below one of the arm openings, such as the first arm opening 26 which has the first loop 40 positioned beneath it, and the loop 50 may be positioned between the arm opening 26 and the lower edge 32. The second loop 50 may also be positioned between the first loop 40 and the lower edge 32 at a location that is suitable for the second loop to receive the lower arm portion 4 of the arm 2 when the upper arm portion 3 is received by the first loop 40.

The second loop 50 may define a second passage 42 through which the lower arm portion 4 is insertable, and the second passage 52 may extend along a substantially vertically-oriented axis when the garment 12 is worn by a standing person such that the lower arm portion 4 is held in a substantially vertical orientation in association with the torso 5. The axes of the first passage 42 and second passage 52 may be substantially vertically aligned with each other when the garment 12 is worn by a standing person.

Like the first loop 40, the second loop 50 may also be configured to open and close. The second loop 50 may open to receive, and release, the lower arm portion 4, and may close to encircle and embrace the lower arm portion 4 to secure the arm portion 4 against movement out of the second loop. Illustratively, the second loop 50 may include a pair of second flaps 54, 55 which are joinable together to close the loop 50, and which are releasable from each other to open the loop 50. The second flaps 54, 55 may have connecting elements 56, 57 thereon to permit releasable connection of the second flaps together, as well as release of the second flaps from each other. Optionally, the second connecting elements 56, 57 may also comprise a hook and loop fasteners.

The arm support elements 38 may also include a third loop 60 which is configured for removably receiving the lower arm portion 4 of the arm of the person, typically as an alternative to the lower arm portion being received in the second loop 50. The third loop 60 may also be mounted on the outer surface 22 of the garment 12 by securing the loop 60 to the garment using a suitable technique.

The third loop 60 may be positioned on the front 14 of the garment 12 and may be positioned between the first lateral side 16 and the slit 34 of the garment. The third loop 60 may be positioned adjacent to the slit 34, and may also be positioned at a vertical level which is medial to, or intermediate between, the vertical levels of the first 40 and second 50 loops when the garment 12 is worn by a standing person. The third loop 60 may define a third passage 62 through which the lower arm portion of the person is insertable. The third passage 62 may extend along a substantially horizontal axis when the garment is worn by a standing person such that the lower arm portion 4 is held in a substantially horizontal orientation. The axis of the third passage 62 is thus oriented substantially perpendicular to the axes of the first 42 and second 52 passages of the respective loops.

Similar to the first and second loops, the third loop 60 may also be configured to be openable to receive, and be closable to encircle, the lower arm portion 4 to secure the arm portion 4 against movement out of the second loop. Illustratively, the third loop 60 may include a pair of third flaps 64, 65 which are joinable together to close the loop 60, and which are releasable from each other to open the loop 60. The third flaps 64, 65 may have connecting elements 66, 67 thereon to permit releasable connection of the third flaps together, as well as release of the third flaps from each other. Optionally, the third connecting elements 66, 67 may also comprise a hook and loop fasteners.

In the first position, the arm of the person may be engaged by the first loop 40 and the second loop 50 such that the upper arm portion is substantially vertically oriented and the lower arm portion is also substantially vertically oriented. In the second position, the arm of the person may be engaged by the first loop 40 and the third loop 60 such that the upper arm portion is substantially vertically oriented and the lower arm portion is substantially horizontally oriented. In a third position, the upper arm portion may be engaged by the first loop 40 but the lower arm portion may be free of engagement by the second and third loops, so that the forearm, wrist and hand are able to move relatively freely and the upper arm portion may rotate at the shoulder to facilitate the range of free movement by the lower arm portion.

It should be appreciated that in the foregoing description and appended claims, that the terms “substantially” and “approximately,” when used to modify another term, mean “for the most part” or “being largely but not wholly or completely that which is specified” by the modified term.

It should also be appreciated from the foregoing description that, except when mutually exclusive, the features of the various embodiments described herein may be combined with features of other embodiments as desired while remaining within the intended scope of the disclosure.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated.

With respect to the above description then, it is to be realized that the optimum dimensional relationships for the parts of the disclosed embodiments and implementations, to include variations in size, materials, shape, form, function and manner of operation, assembly and use, are deemed readily apparent and obvious to one skilled in the art in light of the foregoing disclosure, and all equivalent relationships to those illustrated in the drawings and described in the specification are intended to be encompassed by the present disclosure.

Therefore, the foregoing is considered as illustrative only of the principles of the disclosure. Further, since numerous modifications and changes will readily occur to those skilled in the art, it is not desired to limit the disclosed subject matter to the exact construction and operation shown and described, and accordingly, all suitable modifications and equivalents may be resorted to that fall within the scope of the claims.

I claim:

1. A sling apparatus for supporting an arm of a person with respect to a torso of the person, the arm including an upper arm portion and a lower arm portion, the sling apparatus comprising:
a garment configured to be worn on the torso of the person, the garment having a front for positioning adjacent to a front of the torso, a back for positioning adjacent to a back of the torso, and at least one lateral side extending between the front and back for positioning adjacent to a side of the torso, the garment having at least one arm opening in the at least one lateral side for receiving the arm of the person; and
arm support elements mounted on the garment and being configured to support the arm of the person wearing the garment in at least two positions with respect to the torso;
wherein the at least two positions include a first position of the arm having the elbow extended with a shoulder associated with the arm of the person being in a neutral rotation orientation and a second position of the arm with the elbow flexed and the shoulder associated with the arm being in an adducted, internal rotation position.

2. The apparatus of claim 1 wherein the at least two positions including a third position of the arm having the upper arm portion constrained in a position adjacent to the torso and the lower arm portion is unconstrained.

3. The apparatus of claim 1 wherein the arm support elements include a plurality of loops for receiving portions of the arm of the person wearing the garment to support the arm in each of the at least two positions.

4. The apparatus of claim 3 wherein the plurality of loops is configured to constrain the portions of the arm in each of the two arm positions.

5. The apparatus of claim 3 wherein the plurality of loops includes a first loop for receiving the upper arm portion of the arm of the person in the first position and the second position of the arm, the first loop being mounted on the at least one lateral side of the garment in a position below the at least one arm opening.

6. The apparatus of claim 5 wherein the first loop defines a first passage through which the upper arm portion of the person is insertable, the first passage extending along a substantially vertical axis when the garment is worn by the person in a standing position such that the upper arm portion is held in a substantially vertical orientation.

7. The apparatus of claim 5 wherein the plurality of loops includes a second loop for receiving the lower arm portion of the arm of the person in the first position of the arm, the second loop being mounted on the at least one lateral side of the garment below the first loop.

8. The apparatus of claim 7 wherein the second loop defines a second passage through which the lower arm portion of the person is insertable, the second passage extending along a substantially vertical axis when the garment is worn by the person in a standing position such that the lower arm portion is held in a substantially vertical orientation.

9. The apparatus of claim 8 wherein an axis of the second passage is substantially aligned with the substantially vertical axis of a first passage defined by the first loop.

10. The apparatus of claim 7 wherein the plurality of loops includes a third loop for receiving the lower arm portion of the arm of the person in the second position of the arm, the third loop being mounted on the front of the garment.

11. The apparatus of claim 10 wherein the third loop defines a third passage through which the lower arm portion of the person is insertable, the third passage extending along a substantially horizontal axis when the garment is worn by the person in a standing position such that the lower arm portion is held in a substantially horizontal orientation.

12. The apparatus of claim 11 wherein the garment has a neck opening extending through the garment for receiving the neck of the person, a torso opening opposite of the neck opening, and a slit formed on the front of the garment, the slit extending across the front of the garment between the neck opening and the torso opening; and
wherein the third loop is positioned adjacent to the slit of the garment.

13. The apparatus of claim 3 wherein the first loop is configured to open to release the upper arm portion of the person from the first loop and to accept the upper arm portion into the first loop, and close to encircle and embrace the upper arm portion against movement out of the first loop.

14. The apparatus of claim 13 wherein the first loop includes a pair of first flaps joinable together and releaseable from each other.

15. The apparatus of claim 14 wherein the first flaps have first connecting elements to permit releasable connection of the first flaps together.
